(19) United States
(12) Patent Application Publication
(10) Pub. No.: US 2019/0314159 A1
(43) Pub. Date: Oct. 17, 2019

(54) RADIAL HEAD REPLACEMENT PROSTHESIS

(71) Applicant: RevOrtho LLC, Great Neck, NY (US)

(72) Inventors: Craig Eric Klinger, Great Neck, NY (US); Jose Bernardo Toro Arbelaez, Warwick, NY (US); David Leonard Helfet, New York, NY (US)

(21) Appl. No.: 16/382,557

(22) Filed: Apr. 12, 2019

Related U.S. Application Data
(60) Provisional application No. 62/658,459, filed on Apr. 16, 2018.
(60) Provisional application No. 62/670,756, filed on May 12, 2018.
(60) Provisional application No. 62/788,909, filed on Jan. 6, 2019.

(51) Int. Cl. A61F 2/38; A61F 2/30

(52) U.S. Cl. CPC A61F 2/3804; A61F 2/30749; A61F 2002/3827; A61F 2002/3809; A61F 2002/30172; A61F 2002/30332; A61F 2002/3038; A61F 2002/30784; A61F 2002/30405

(57) ABSTRACT

The presented disclosure is a prosthesis for performing radial head replacement in the setting of fractures or degenerative conditions of the radial head, and other conditions including avascular necrosis. The presented radial head replacement prostheses can be implanted in a cement-less or cemented fashion. The prosthesis of the presented disclosure comprises a radial head replacement which in various embodiments includes (1) one or more interlocking screw holes along the stem, (2) a radial head replacement prosthesis with a two component design and with a tapered connection, (3) a radial head replacement prosthesis with a two component design and with a cylindrical connection, (4) a radial head replacement prosthesis with a two component design and with a curved stem, (5) a radial head replacement prosthesis with a two component design and with an inter-component screw connection, and (6) a radial head replacement prosthesis with a one-piece design.

### CROSS-REFERENCE TO RELATED APPLICATIONS

The present application claims the benefit of U.S. Provisional Patent Application Ser. No. 62/658,459 filed on Apr. 16, 2018, U.S. Provisional Patent Application Ser. No. 62/670,756 filed on May 12, 2018, and U.S. Provisional Patent Application Ser. No. 62/788,909 filed on Jan. 6, 2019 by the present inventors, the entire contents of each of which is incorporated herein by reference thereto.

### FIELD OF THE DISCLOSURE

The present disclosure relates to radial head replacement.

### BACKGROUND

Implants may be used, e.g., when one or more human (or animal) body parts need replacing. For example, known radial head replacement implants may have been developed, to treat such problems within the elbow joint as, e.g., fractures or degenerative conditions of the radial head including avascular necrosis.

The present orthopedic implants relate generally to prosthetic implants involving the elbow and more specifically, to a radial head replacement prosthesis which can be fitted to a prepared and shaped proximal radius and implanted with or without the use of bone cement, as one or more undersurfaces may be porous in nature.

Various prostheses have been proposed for radial head replacement. See, for example, U.S. Pat. Nos. 6,709,459, 7,608,110, 8,353,965, and 9,636,228. However, despite numerous prostheses design implementations, a high rate of implant loosening is still reported with radiographic loosening rates as high a 40% (see, Levy J. C., Formaini N. T., Kurowicki J.: Outcomes and radiographic findings of anatomic press-fit radial head arthroplasty., Journal of shoulder and elbow surgery., 25(5):802-809, 2016).

### SUMMARY

The radial head replacement prosthesis according to an example embodiment of the present disclosure allows for multiple fixation options. Various fixation options are afforded with a prosthesis allowing screw engagement from the outer proximal radial cortex into the prosthesis stem, and a two-piece modular radial head replacement prosthesis with tapered connection affording an interference fit between components. These fixation options provide for a range of scalable fixation to allow implant fixation to be addressed based on bone quality. Other implantation options include a use of a curved stem. Furthermore, various component connections are available including a tapered connection with or without the presence of anti-rotation pegs, a cylindrical connection for inter-component rotational capability, an inter-component screw connection, and/or a one-piece radial head replacement.

In one or more embodiments of the present disclosure, a radial head replacement prothesis may include a radial head component with a concave bearing surface configured to articulate with a capitelum of a distal humerus and a stem component with at least one interlocking screw hole configured to accept a screw.

One or more of the following features may be included. The radial head replacement may include a tapered connection may be configured to couple the radial head component and the stem component. The stem component may include an alignment indentation. The radial head component may include one or more anti-rotation holes. The stem component may include one or more anti-rotation pegs. The radial head replacement may include a cylindrical connection configured to couple the radial head component and the stem component. The stem component may be a tapered curved stem. The radial head component and the stem component may be a single component. The radial head component and the stem component may each be configured to accommodate an inter-component partially threaded screw configured to couple the radial head component and the stem component. The stem component may include at least one surface with a porous coating.

In one or more embodiments of the present disclosure, a radial head replacement prothesis may include a radial head component with a concave bearing surface configured to articulate with a capitelum of a distal humerus, a tapered receptacle, a stem component with at least one interlocking screw hole configured to accept a screw and a tapered extension configured to couple to the tapered receptacle of the radial head component.

One or more of the following features may be included. The stem component may include an alignment indentation. The radial head component may include one or more anti-rotation holes. The stem component may include one or more anti-rotation pegs. The radial head replacement may include a cylindrical connection configured to couple the radial head component and the stem component. The stem component may be a tapered curved stem. The radial head component and the stem component may be a single component. The radial head component and the stem component may each be configured to accommodate an inter-component partially threaded screw configured to couple the radial head component and the stem component. The stem component may include at least one surface with a porous coating.

In one or more embodiments of the present disclosure, a radial head replacement prothesis may include a radial head component with a concave bearing surface configured to articulate with a capitelum of a distal humerus, a cylindrical receptacle, a stem component with at least one interlocking screw hole configured to accept a screw and a cylindrical extension configured to couple to the cylindrical receptacle of the radial head component.

One or more of the following features may be included. The radial head component may include one or more anti-rotation holes. The stem component may include one or more anti-rotation pegs. The stem component may be a tapered curved stem. The stem component may include at least one surface with a porous coating.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1A is a front view illustrating a radial head component with tapered receptacle according to one or more example implementations of the disclosure;

FIG. 1B is a right view illustrating the radial head component with tapered receptacle according to one or more example implementations of the disclosure;

FIG. 1C is a top view illustrating the radial head component with tapered receptacle according to one or more example implementations of the disclosure;

FIG. 1D is a bottom view illustrating the radial head component with tapered receptacle according to one or more example implementations of the disclosure;

FIG. 1E is a cross-sectional front view illustrating the radial head component with tapered receptacle according to one or more example implementations of the disclosure;

FIG. 1F is a bottom perspective view illustrating the radial head component with tapered receptacle according to one or more example implementations of the disclosure;

FIG. 2A is a front view illustrating a stem component with tapered extension according to one or more example implementations of the disclosure;

FIG. 2B is a right view illustrating the stem component with tapered extension according to one or more example implementations of the disclosure;

FIG. 2C is a top view illustrating the stem component with tapered extension according to one or more example implementations of the disclosure;

FIG. 2D is a bottom view illustrating the stem component with tapered extension according to one or more example implementations of the disclosure;

FIG. 2E is a cross-sectional front view illustrating the stem component with tapered extension according to one or more example implementations of the disclosure;

FIG. 2F is a perspective view illustrating the stem component with tapered extension according to one or more example implementations of the disclosure;

FIG. 2G is a right view illustrating a radial head replacement prosthesis with tapered connection assembly according to one or more example implementations of the disclosure;

FIG. 2H is a cross-sectional front view illustrating the radial head replacement prosthesis with tapered connection assembly according to one or more example implementations of the disclosure;

FIG. 2I is a perspective view illustrating the radial head replacement prosthesis with tapered connection assembly according to one or more example implementations of the disclosure;

FIG. 3A is a front view illustrating the radial head replacement prosthesis with tapered connection assembly within an elbow following implantation with two screws according to one or more example implementations of the disclosure;

FIG. 3B is a right view illustrating the radial head replacement prosthesis with tapered connection assembly within an elbow following implantation with two screws according to one or more example implementations of the disclosure;

FIG. 4A is a front view illustrating a stem component with tapered extension and alignment indentation according to one or more example implementations of the disclosure;

FIG. 4B is a right view illustrating the stem component with tapered extension and alignment indentation according to one or more example implementations of the disclosure;

FIG. 4C is a top view illustrating the stem component with tapered extension and alignment indentation according to one or more example implementations of the disclosure;

FIG. 4D is a bottom view illustrating the stem component with tapered extension and alignment indentation according to one or more example implementations of the disclosure;

FIG. 4E is a cross-sectional front view illustrating the stem component with tapered extension and alignment indentation according to one or more example implementations of the disclosure;

FIG. 4F is a perspective view illustrating the stem component with tapered extension and alignment indentation according to one or more example implementations of the disclosure;

FIG. 5A is a front view illustrating a radial head component with tapered receptacle and anti-rotation holes according to one or more example implementations of the disclosure;

FIG. 5B is a right view illustrating the radial head component with tapered receptacle and anti-rotation holes according to one or more example implementations of the disclosure;

FIG. 5C is a top view illustrating the radial head component with tapered receptacle and anti-rotation holes according to one or more example implementations of the disclosure;

FIG. 5D is a bottom view illustrating the radial head component with tapered receptacle and anti-rotation holes according to one or more example implementations of the disclosure;

FIG. 5E is a cross-sectional front view illustrating the radial head component with tapered receptacle and anti-rotation holes according to one or more example implementations of the disclosure;

FIG. 5F is a bottom perspective view illustrating the radial head component with tapered receptacle and anti-rotation holes according to one or more example implementations of the disclosure;

FIG. 6A is a front view illustrating a stem component with tapered extension and anti-rotation pegs according to one or more example implementations of the disclosure;

FIG. 6B is a right view illustrating the stem component with tapered extension and anti-rotation pegs according to one or more example implementations of the disclosure;

FIG. 6C is a top view illustrating the stem component with tapered extension and anti-rotation pegs according to one or more example implementations of the disclosure;

FIG. 6D is a bottom view illustrating the stem component with tapered extension and anti-rotation pegs according to one or more example implementations of the disclosure;

FIG. 6E is a cross-sectional front view illustrating the stem component with tapered extension and anti-rotation pegs according to one or more example implementations of the disclosure;

FIG. 6F is a perspective view illustrating the stem component with tapered extension and anti-rotation pegs according to one or more example implementations of the disclosure;

FIG. 6G is a right view illustrating a radial head replacement prosthesis with tapered connection and anti-rotation pegs assembly according to one or more example implementations of the disclosure;

FIG. 6H is a cross-sectional front view illustrating the radial head replacement prosthesis with tapered connection and anti-rotation pegs assembly according to one or more example implementations of the disclosure;

FIG. 6I is a perspective view illustrating the radial head replacement prosthesis with tapered connection and anti-rotation pegs assembly according to one or more example implementations of the disclosure;

FIG. 7A is a front view illustrating a radial head component with cylindrical receptacle according to one or more example implementations of the disclosure;

FIG. 7B is a right view illustrating the radial head component with cylindrical receptacle according to one or more example implementations of the disclosure;

FIG. 7C is a top view illustrating the radial head component with cylindrical receptacle according to one or more example implementations of the disclosure;

FIG. 7D is a bottom view illustrating the radial head component with cylindrical receptacle according to one or more example implementations of the disclosure;

FIG. 7E is a cross-sectional front view illustrating the radial head component with cylindrical receptacle according to one or more example implementations of the disclosure;

FIG. 7F is a bottom perspective view illustrating the radial head component with cylindrical receptacle according to one or more example implementations of the disclosure;

FIG. 8A is a front view illustrating a stem component with cylindrical extension according to one or more example implementations of the disclosure;

FIG. 8B is a right view illustrating the stem component with cylindrical extension according to one or more example implementations of the disclosure;

FIG. 8C is a top view illustrating the stem component with cylindrical extension according to one or more example implementations of the disclosure;

FIG. 8D is a bottom view illustrating the stem component with cylindrical extension according to one or more example implementations of the disclosure;

FIG. 8E is a cross-sectional front view illustrating the stem component with cylindrical extension according to one or more example implementations of the disclosure;

FIG. 8F is a perspective view illustrating the stem component with cylindrical extension according to one or more example implementations of the disclosure;

FIG. 8G is a right view illustrating a radial head replacement prosthesis with cylindrical connection assembly according to one or more example implementations of the disclosure;

FIG. 8H is a cross-sectional front view illustrating the radial head replacement prosthesis with cylindrical connection assembly according to one or more example implementations of the disclosure;

FIG. 8I is a perspective view illustrating the radial head replacement prosthesis with cylindrical connection assembly according to one or more example implementations of the disclosure;

FIG. 9A is a front view illustrating a stem component with tapered extension and curved stem according to one or more example implementations of the disclosure;

FIG. 9B is a right view illustrating the stem component with tapered extension and curved stem according to one or more example implementations of the disclosure;

FIG. 9C is a top view illustrating the stem component with tapered extension and curved stem according to one or more example implementations of the disclosure;

FIG. 9D is a bottom view illustrating the stem component with tapered extension and curved stem according to one or more example implementations of the disclosure;

FIG. 9E is a cross-sectional front view illustrating the stem component with tapered extension and curved stem according to one or more example implementations of the disclosure;

FIG. 10A is a front view illustrating a one-piece radial head replacement prosthesis according to one or more example implementations of the disclosure;

FIG. 10B is a right view illustrating the one-piece radial head replacement prosthesis according to one or more example implementations of the disclosure;

FIG. 10C is a top view illustrating the one-piece radial head replacement prosthesis according to one or more example implementations of the disclosure;

FIG. 10D is a bottom view illustrating the one-piece radial head replacement prosthesis according to one or more example implementations of the disclosure;

FIG. 10E is a cross-sectional front view illustrating the one-piece radial head replacement prosthesis according to one or more example implementations of the disclosure;

FIG. 10F is a cross-sectional front view illustrating the one-piece radial head replacement prosthesis according to one or more example implementations of the disclosure;

FIG. 11A is a front view illustrating a radial head component with inter-component screw hole according to one or more example implementations of the disclosure;

FIG. 11B is a right view illustrating the radial head component with inter-component screw hole according to one or more example implementations of the disclosure;

FIG. 11C is a top view illustrating radial head component with inter-component screw hole according to one or more example implementations of the disclosure;

FIG. 11D is a bottom view illustrating the radial head component with inter-component screw hole according to one or more example implementations of the disclosure;

FIG. 11E is a cross-sectional front view illustrating the radial head component with inter-component screw hole according to one or more example implementations of the disclosure;

FIG. 11F is a bottom perspective view illustrating the radial head component with inter-component screw hole according to one or more example implementations of the disclosure;

FIG. 12A is a front view illustrating a stem component with inter-component screw hole according to one or more example implementations of the disclosure;

FIG. 12B is a right view illustrating the stem component with inter-component screw hole according to one or more example implementations of the disclosure;

FIG. 12C is a top view illustrating the stem component with inter-component screw hole according to one or more example implementations of the disclosure;

FIG. 12D is a bottom view illustrating the stem component with inter-component screw hole according to one or more example implementations of the disclosure;

FIG. 12E is a cross-sectional front view illustrating the stem component with inter-component screw hole according to one or more example implementations of the disclosure;

FIG. 12F is a perspective view illustrating the stem component with inter-component screw hole according to one or more example implementations of the disclosure;

FIG. 12G is a front view illustrating an inter-component partially threaded screw according to one or more example implementations of the disclosure;

FIG. 12H is a perspective view illustrating the inter-component partially threaded screw according to one or more example implementations of the disclosure;

FIG. 12I is a right view illustrating a radial head replacement prosthesis with inter-component screw connection assembly according to one or more example implementations of the disclosure;

FIG. 12J is a cross-sectional front view illustrating the radial head replacement prosthesis with inter-component screw connection assembly according to one or more example implementations of the disclosure; and

FIG. 12K is a perspective view illustrating the radial head replacement prosthesis with inter-component screw connection assembly according to one or more example implementations of the disclosure.

Like reference symbols in the various drawings indicate like elements.

### DETAILED DESCRIPTION

### Reference Numerals

9—radial head component with tapered receptacle
10—articular concavity
12—tapered receptacle
20—stem component with tapered extension
22—tapered stem
26—tapered extension
29—planar undersurface
30—interlocking screw hole
32—flute
34—radial head replacement prosthesis with tapered connection assembly
35—capitellum
36—ulna
37—proximal radius
38—screw
40—stem component with tapered extension and alignment indentation
44—alignment indentation
50—radial head component with tapered receptacle and one or more anti-rotation holes
52—anti-rotation holes
60—stem component with tapered extension and one or more anti-rotation pegs
61—Anti-rotation peg
62—radial head replacement prosthesis with tapered connection and anti-rotation pegs assembly
70—radial head component with cylindrical receptacle
72—cylindrical receptacle
80—stem component with cylindrical extension
82—cylindrical extension
84—radial head replacement prosthesis with cylindrical connection assembly
90—stem component with tapered extension and curved stem
92—tapered curved stem
100—one-piece radial head replacement prosthesis
102—upper aspect
104—lower aspect with tapered stem
110—radial head component with inter-component screw hole
112—recessed inter-component threaded screw hole
114—cube shaped receptacle
116—screw hole extension
120—stem component with inter-component screw hole
122—stem inter-component non-threaded screw hole
124—cube shaped extension
126—inter-component partially threaded screw
128—threaded aspect
130—non-threaded aspect
131—tapered aspect
132—radial head replacement prosthesis with inter-component screw connection assembly

Implants may be used, e.g., when one or more human (or animal) body parts need replacing. For example, known radial head replacement implants may have been developed, to treat such problems within the elbow joint as, e.g., fractures or degenerative conditions of the radial head. Radial head replacement prostheses include a component or components which provide a bearing surface for articulation with the capitellum of the distal humerus. The capitellum includes cartilage along its surface for articulation with the native radial head. However, such implants of the prior art may be associated with complications, especially, aseptic loosening, which may then require secondary revision surgeries.

As will be discussed in greater detail below, the example radial head replacement prosthesis provides one or more fixation options. Fixation options of the radial head replacement prosthesis of the present disclosure include the capability for stem engagement by either single or dual screws placed from the outer cortex into interlocking screw holes along the prosthesis stem in either un-cemented or cemented fashion. These fixation options may provide for a range of scalable fixation in order to allow the surgeon to increase fixation when poor bone quality is encountered and/or depending on the severity of an associated radial head fracture or degenerative condition of the radial head. Use of these fixation options may lead to decreases in incidence of aseptic loosening in radial head replacement procedures as related to the radial head replacement prosthesis of the present disclosure. In some embodiments, the radial head replacement prosthesis can be fitted to the prepared and shaped proximal radius and implanted with or without the use of bone cement, as one or more undersurfaces may be porous in nature to allow for osseous integration with the underlying bone. Additionally, in various embodiments, options allow the radial head component to be retained through the use of a tapered inter-component connection with or without the presence of anti-rotation pegs, a cylindrical connection for inter-component articulation, a tapered connection with a curved stem, and a one-piece radial head replacement prosthesis in which the radial head and tapered stem aspects may be joined or coupled during manufacture producing a radial head with a fixed stem.

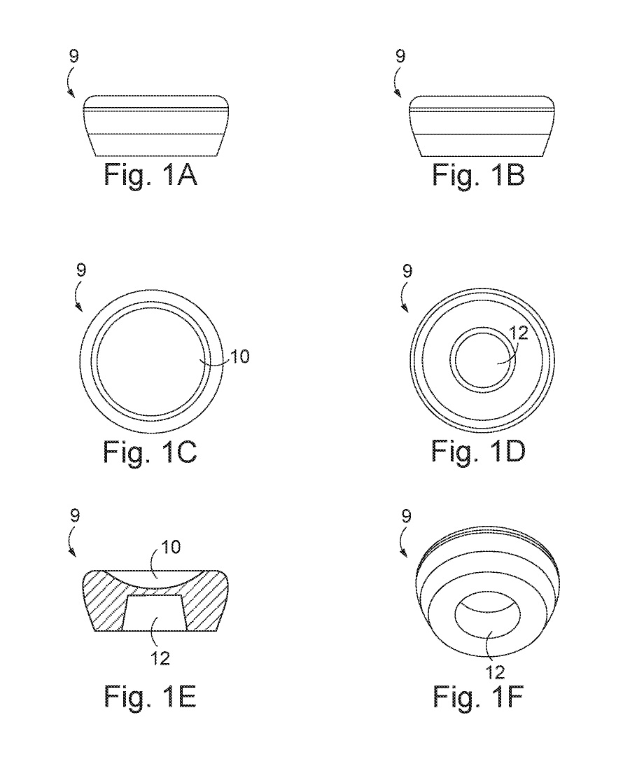

Referring to the example embodiments of FIGS. 1A-1F, FIGS. 1A and 1B are a front and right views, respectively, illustrating a radial head component with tapered receptacle 9 in accordance with one exemplary embodiment of the radial head replacement prosthesis of the present disclosure. The radial head component with tapered receptacle 9 is one component of a radial head replacement prosthesis with a two-component design. FIGS. 1C and 1D are top and bottom views, respectively, further illustrating radial head component with tapered receptacle 9 including articular concavity 10, which may be centered along the top surface and may be highly polished and may provide a smooth bearing surface for the native capitellum. This bearing surface may be concave in shape matching the concave nature of the native radial head articular surface. Also included along the bottom aspect is tapered receptacle 12 which may be included to allow joining of radial head replacement prosthesis components through an interference fit. Tapered receptacle 12 may be located centrally along the bottom aspect and provides a connection point using a standard Morse taper, as known in the art. The Morse taper connection allows for an interference fit and cold-welding between two components with compression during implantation and through physiologic loading. The Morse taper connection includes two differing taper angles components creating a mismatch and allowing for an interference fit for cold-welding between two components with compression during implantation and through physiologic loading. FIGS. 1E and 1F is a cross-sectional front view and a bottom perspective view, respectively, further illustrating the radial head component with articular concavity 10 and tapered receptacle 12. Tapered receptacle 12 has a diameter which progressively decreases from its lower aspect to its upper aspect. Radial head component with tapered receptacle 9 may be made in multiple sizes to accommodate variation in anatomy variability and amount of bone resected during the operative procedure. Versions include varying diameters and heights of the radial head component with tapered receptacle 12. The diameter and dimensions of tapered receptacle 12 may be consistent throughout all sizes in order to allow for modularity and compatibility. The radial head component with tapered receptacle 9 may therefore come in multiple sizing options to enable the surgeon to select appropriate size implants for each individual patient, as needed. For example, patients with larger anatomy can receive a proportionally larger radial head component.

In some embodiments, the radial head component with tapered receptacle may be manufactured to consist of metal or metal alloy including a high carbon cobalt chrome (CoCr) alloy. In some embodiments, it may also be made of an alternate metal or metal alloy including stainless steel, titanium, or another suitable material including surgical-grade ceramics, plastics, rubber, or combination materials. It can be formed as cast, forged, milled or drilled through machining processes or a combination of methods. Standard machining techniques can be used during component manufacture including for creation of the tapered receptacle. The radial head component with tapered receptacle may be manufactured in constituent form and later joined or coupled through welding or other suitable techniques. The radial head component with tapered receptacle including articular concavity 10 and additional surfaces may be further smoothed to achieve a highly-polished finish during production through standard manufacturing techniques, as known in the art. The tapered receptacle may have a rough surface.

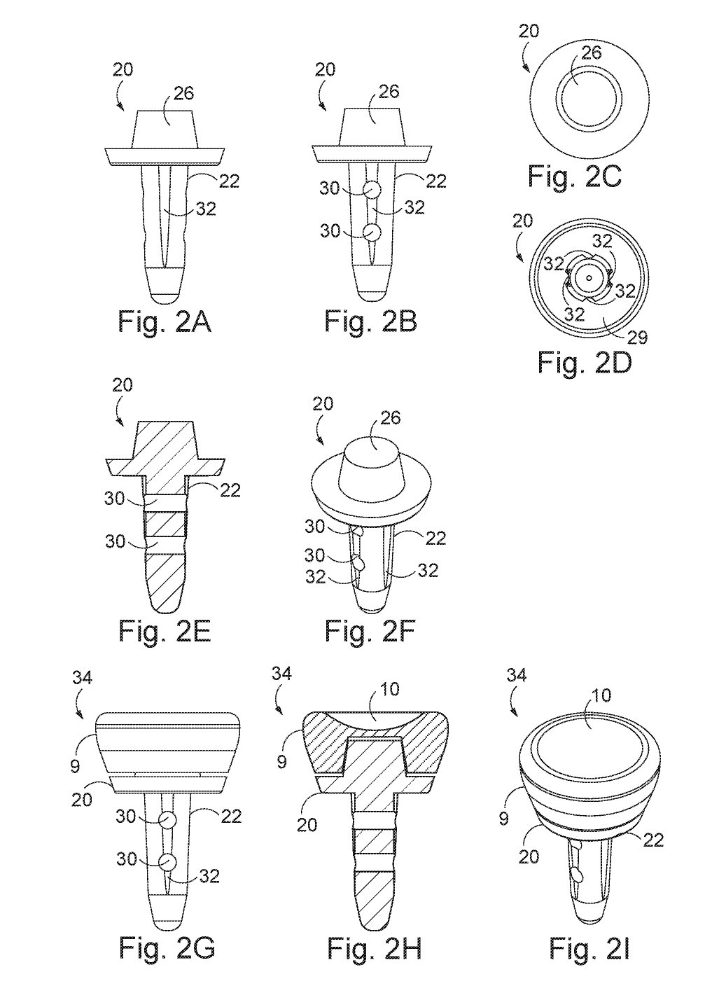

Referring also to the example embodiments of FIGS. 2A-2I, FIGS. 2A and 2B are a front and right views, respectively, illustrating a stem component with tapered extension 20 in accordance with one exemplary embodiment of the radial head replacement prosthesis of the present disclosure. The stem component with tapered extension 20 is one component of a radial head replacement prosthesis with a two-component design. The stem component with tapered extension 20 includes tapered extension 26. Tapered extension 26 has a diameter which progressively decreases from its lower aspect to its upper aspect. Tapered extension 26 may provide a connection point for the radial head component with tapered receptacle 9 using a standard Morse taper, as known in the art, which may provide for an interference fit. In some embodiments, the stem component with tapered extension 20 includes tapered stem 22, with inclusion of a taper for easier implantation. Also included in the lower aspect in duplicate is interlocking screw hole 30 which may accept screws. A screw may engage interlocking screw hole 30 from the cortical bone. Also included is tapered stem 22 is four of flute 32. The flutes are included to further aid implantation and provide for additional rotational stability. FIGS. 2C and 2D are top and bottom views, respectively, further illustrating the stem component with tapered extension 20 including tapered extension 26, planar undersurface 29, and four of flute 32. FIGS. 2E and 2F are a cross-sectional front view and a perspective view, respectively, further illustrating the stem component with tapered extension 20 including tapered extension 26, tapered stem 22, and interlocking screw hole 30 present in duplicate. FIGS. 2G and 2H are a right view and a cross-sectional front view, respectively, illustrating a radial head replacement prosthesis with tapered connection assembly 34 including radial head component with tapered receptacle 9 and stem component with tapered extension 20. In some embodiments, the radial head component with tapered receptacle may be seated onto the stem component with tapered extension to achieve an interference fit with tapered extension 26 inserting into the tapered receptacle 12. FIG. 2I is a perspective view further illustrating the radial head replacement prosthesis with tapered connection assembly 34 including final seating of radial head component with tapered receptacle 9 onto stem component with tapered extension 20.

The stem component with tapered extension may be made in multiple sizes to accommodate anatomical variations. Embodiments of tapered stem 22 may include varying diameters and lengths. The diameter and dimensions of the tapered extension may be consistent throughout all sizes in order to allow for modularity and compatibility. The stem component with tapered extension may therefore come in multiple sizing options to enable the surgeon to select appropriate size implants for each individual patient, as needed. For example, patients with larger anatomy can receive a proportionally larger stem component with tapered extension.

The stem component with tapered extension may be manufactured to consist of metal or metal alloy including a high carbon cobalt chrome (CoCr) alloy. It may also be made of an alternate metal or metal alloy including stainless steel, titanium, or another suitable material including surgical-grade ceramics, plastics, rubber, or combination materials. It can be formed as cast, forged, milled or drilled through machining processes or a combination of methods. Standard machining techniques can be used during component manufacture including for creation of the tapered extension. The stem component with tapered extension may also be manufactured in constituent form and later joined or coupled through welding or other suitable techniques. Surfaces may be further smoothed to achieve a highly-polished finish during production through standard manufacturing techniques, as known in the art. The planar undersurface 29 and lower segment 22, including each flute 32, may include a porous, textured, granular and/or beaded surface coating as known in the art. The tapered extension may have a rough surface.

The radial head replacement prosthesis with tapered connection assembly 34 of the present embodiment may be implanted through surgical techniques for radial head replacement, as known in the prior art with some slight modifications. The patient may be positioned supine on the operating table and the operative upper extremity may be prepared and draped to allow access from the axilla to the hand allowing for elbow flexion, extension and rotation during the radial head replacement procedure. A lateral surgical approach to the elbow may be performed to allow access to the radial head and care is taken to avoid the deep branch of the radial nerve anterior of the capsule during the procedure. The lateral collateral ligament may also be protected during the procedure by maintaining the capsule incision in front of the anterior aspect of the anconeus muscle and parallel to the extensor carpi ulnaris fascia limit. The annular ligament may be opened laterally to allow for visualization and preparation of the radial head. Further exposure of the proximal radial cortex can be performed with periosteal elevator or bladed instrument. A lateral epicondyle osteotomy can be performed if needed to provide for extensile surgical access. Next, the native radial head may be resected using e.g., a micro-sagittal saw at the level of the radial neck and perpendicular to the axis of the neck to allow for optimal implant seating, as known in the art. Then the radial canal may be opened using a canal finder, as known in the art. Next a surgical drill or broach/es may be used, as known in the art, to create a medullary channel to accommodate the tapered stem. Following channel preparation, the appropriate stem component with tapered extension 20 may be selected to match the diameter of the medullary channel and desired stem length. The stem component with tapered extension 20 may be implanted by insertion into the prepared medullary channel until fully seated with the planar undersurface 29 abutting the osteotomized bone of the radial cortex. Bone cement may be inserted in the medullary canal prior to implantation but is not preferred. In preparation for insertion of the interlocking screws for implant fixation, a periosteal elevator can be used with additional retraction along the radial cortex as needed. One or both of interlocking screw hole 30 may be engaged with a short screw inserted from the outer cortex entering and engaging the interlocking screw hole and may extend beyond the interlocking screw hole but not beyond the far cortex. Intraoperative fluoroscopic image guidance can aid localization and drilling of these cortical holes to accommodate the screws to engage the interlocking screw holes within the tapered stem. Following implantation of the stem component with tapered extension, an appropriately sized radial head component with tapered receptacle 9 may selected based on the diameter and height of the excised native radial head. The selected radial head component with tapered receptacle 9 may be firmly seated onto the stem component with tapered extension 20 to achieve an interference fit with tapered extension 26 inserting into the tapered receptacle 12. A surgical tamp may be gently tapped along the top periphery of radial head component with tapered receptacle to further aid final seating of the radial head component implant. An advantage of the present embodiment is that the modular design allows for ease of implantation as screw fixation for the stem component with tapered extension is able to be completed prior to sizing and selection of the radial head component with tapered receptacle. The capsule, deep tissue and superficial tissues are then closed and sutured, sequentially, as known in the art.

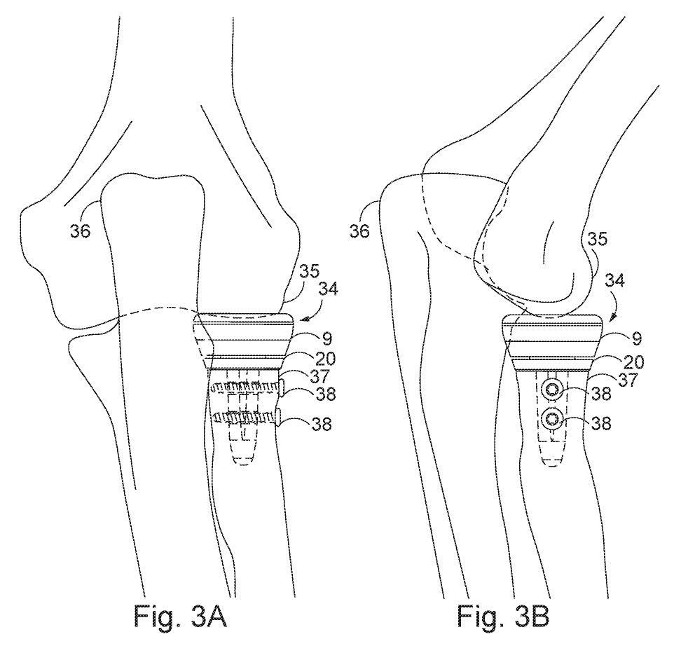

Referring also to the example of FIGS. 3A-3B, FIGS. 3A and 3B are a front and a right view, respectively, further illustrating radial head replacement prosthesis with tapered connection assembly 34 within an elbow following implantation with two screws. Anatomical structures of the elbow are also illustrated including the capitellum 35, ulna 36 and proximal radius 37. Radial head replacement prosthesis with tapered connection assembly 34 includes radial head component with tapered receptacle 9 and stem component with tapered extension 20. Stem component with tapered extension 20 may be secured to the proximal radius 37 using two of screw 38 to obtain stabile fixation. However, it will be appreciated that any type of fastener and any number of fasteners may be used to obtain stable fixation within the scope of the present disclosure.

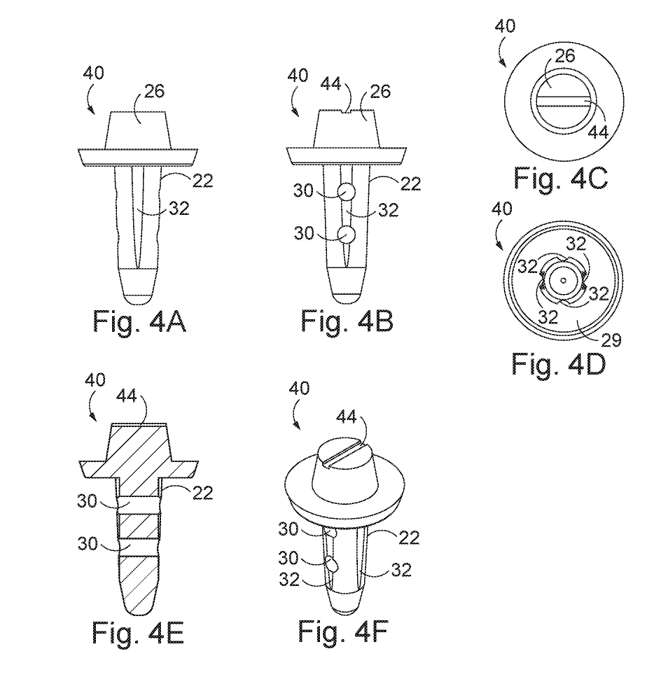

Referring also to the example embodiments of FIGS. 4A-4F, FIGS. 4A and 4B are a front and right views, respectively, illustrating a stem component with tapered extension and alignment indentation 40 in accordance with one exemplary embodiment of the radial head replacement prosthesis of the present disclosure. The stem component with tapered extension and alignment indentation 40 is one component of a radial head replacement prosthesis with a two-component design. The prosthesis and steps for implantation may be the same as that illustrated and described in FIGS. 2A to 2F with a few differences, which are described below. The stem component with tapered extension and alignment indentation 40 may include alignment indentation 44 along the top surface of tapered extension 26. The alignment indentation may be included to provide a linear reference marking and visual aid for prosthesis orientation during implantation. Also included is tapered stem 22, four of flute 32, and e.g., two of interlocking screw holes 30. FIGS. 4C and 4D are top and bottom views, respectively further illustrating the stem component with tapered extension and alignment indentation 40 including alignment indentation 44, planar undersurface 29, and four of flute 32. FIGS. 4E and 4F are a cross-sectional front view and a perspective view, respectively, further illustrating the stem component with tapered extension and alignment indentation 40, including alignment indentation 44, tapered stem 22, and interlocking screw hole 30 present in duplicate. The radial head component with tapered receptacle 9 of FIGS. 1A to 1F may be seated onto stem component with tapered extension and alignment indentation 40 during the operative procedure.

The steps for implantation may generally be the same as illustrated and described in FIGS. 1A to 1F and FIGS. 2A to 2F with a few differences described below. Following surgical preparation including removal of the radial head and preparation of the medullary canal, the stem component with tapered extension and alignment indentation 40 may inserted and seated into the medullary canal with the planar undersurface 29 abutting the osteotomized bone of the radial cortex. One or both of interlocking screw hole 30 may be engaged with a short screw inserted from the outer cortex entering and engaging the interlocking screw hole and may extend beyond the interlocking screw hole but not beyond the far cortex. Intraoperative fluoroscopic image guidance can aid localization and drilling of these cortical holes to accommodate the screws to engage the interlocking screw holes within the tapered stem. Additionally, the alignment indentation 44 of the present embodiment can be used as a visual aid for orientation and localization during the drilling of these cortical holes. Following implantation of the stem component with tapered extension and alignment indentation 40, an appropriately sized radial head component with tapered receptacle 9 may be selected based on the diameter and height of the excised native radial head. The selected radial head component with tapered receptacle may be firmly seated onto the stem component with tapered extension and alignment indentation 40 to achieve an interference fit with tapered extension 26 inserting into the tapered receptacle 12. In some embodiments, a surgical tamp may be gently tapped along the top periphery of the radial head component with tapered receptacle 9 to further aid final seating of the radial head component implant. An advantage of the present embodiment is that inclusion of alignment indentation 44 provides a linear reference marking and visual aid for prosthesis orientation during surgical implantation.

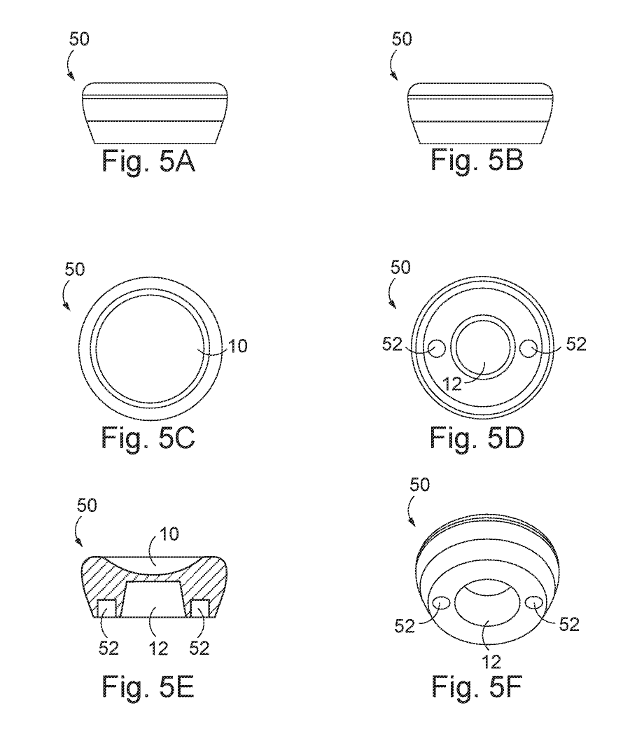

Referring also to the example embodiments of FIGS. 5A-5F, FIGS. 5A and 5B are a front and right views, respectively, illustrating a radial head component with tapered receptacle and one or more anti-rotation holes 50 in accordance with one exemplary embodiment of the radial head replacement prosthesis of the present disclosure. The radial head component with tapered receptacle and one or more anti-rotation holes 50 may be one component of a radial head replacement prosthesis with a two-component design. The prosthesis and steps for implantation are the same as that illustrated and described in FIGS. 1A to 1F with a few differences, which are described below. FIGS. 5C and 5D are top and bottom views, respectively, illustrating tapered receptacle and one or more anti-rotation holes 50 including articular concavity 10, which is centered along the top surface. Included in duplicate along the bottom periphery of the radial head component is anti-rotation hole 52 which provides for additional rotational stability after final implantation. Also included and centered along the bottom surface is tapered receptacle 12. FIGS. 5E and 5F are a cross-sectional front view and bottom perspective view, respectively, further illustrating the radial head component with tapered receptacle and one or more anti-rotation holes 50 including articular concavity 10, anti-rotation hole 52 present in duplicate, and tapered receptacle 12. The radial head component with tapered receptacle and one or more anti-rotation holes 50 is made in multiple sizes to accommodate anatomical variations. Embodiments may include varying diameters and heights of the radial head component with tapered receptacle and one or more anti-rotation holes 50. The diameter and dimensions of the tapered receptacle and each anti-rotation hole may be consistent throughout all sizes in order to allow for modularity and compatibility.

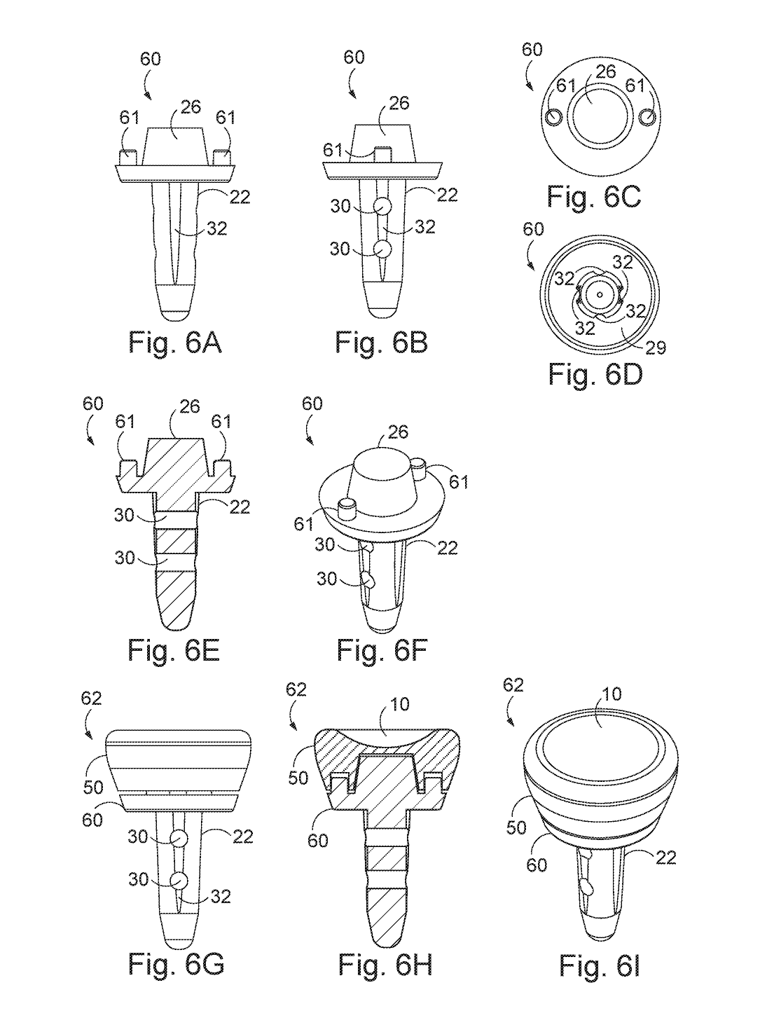

Referring also to the example embodiments of FIG. 6A-6I, FIGS. 6A and 6B are a front and right views, respectively, illustrating a stem component with tapered extension and one or more anti-rotation pegs 60 in accordance with one exemplary embodiment of the radial head replacement prosthesis of the present disclosure. In one embodiment, the stem component with tapered extension and one or more anti-rotation pegs 60 may be one component of a radial head replacement prosthesis with a two-component design. The prosthesis and steps for implantation may generally be the same as that illustrated and described in FIGS. 2A to 2I with a few differences, which are described below. The stem component with tapered extension and one or more anti-rotation pegs 60 may include tapered extension 26 and two of anti-rotation peg 61. The anti-rotation pegs may provide for additional rotational stability when mated to the corresponding radial head component with tapered receptacle and anti-rotation holes 50 and after final implantation. Also included is tapered stem 22, two of interlocking screw hole 30, and four of flute 32. FIGS. 6C and 6D are top and bottom views, respectively, further illustrating the stem component with tapered extension and one or more anti-rotation pegs 60 including Morse tapered extension 26, two of anti-rotation peg 61, four of flute 32 and planar undersurface 29. FIGS. 6E and 6F are a cross-sectional front view and a perspective view, respectively, further illustrating the stem component with tapered extension and one or more anti-rotation pegs 60, including two of anti-rotation peg 61, tapered stem 22, and interlocking screw hole 30 present in duplicate. FIGS. 6G and 6H are a right view and a cross-sectional front view, respectively, illustrating a radial head replacement prosthesis with tapered connection and anti-rotation pegs assembly 62 including radial head component with tapered receptacle and one or more anti-rotation holes 50 and stem component with tapered extension and one or more anti-rotation pegs 60. In this embodiment, the radial head component with tapered receptacle and one or more anti-rotation holes 50 may be seated onto the stem component with tapered extension and one or more anti-rotation pegs 60 to achieve an interference fit with the anti-rotation pegs inserting into the anti-rotation holes, and with the tapered extension inserting into the Morse tapered receptacle. FIG. 6I is a perspective view further illustrating final seating of radial head component with tapered receptacle and one or more anti-rotation holes 50 onto stem component with tapered extension and one or more anti-rotation pegs 60.

The steps for implantation may generally be the same as illustrated and described in FIG. 1A to 1F and FIGS. 2A to 2I, with a few differences described below. Following implantation of the stem component with tapered extension and one or more anti-rotation pegs 60, an appropriately sized radial head component with tapered receptacle and one or more anti-rotation holes 50 may be selected based on the diameter and height of the excised native radial head. The radial head component with tapered receptacle and one or more anti-rotation holes 50 may be oriented to line up with and seat onto the stem component with tapered extension and one or more anti-rotation pegs 60 with the anti-rotation pegs inserting into the anti-rotation holes achieving a shape fit, and with the tapered extension inserting and seating into the tapered receptacle achieving an interference fit. A surgical tamp may be gently tapped along the top periphery of the radial head component with tapered receptacle and one or more anti-rotation holes 50 to further aid final seating of the radial head component implant. An advantage of the present embodiment is that inclusion of anti-rotation peg 61 in duplicate may provide for additional rotational stability after final implantation.

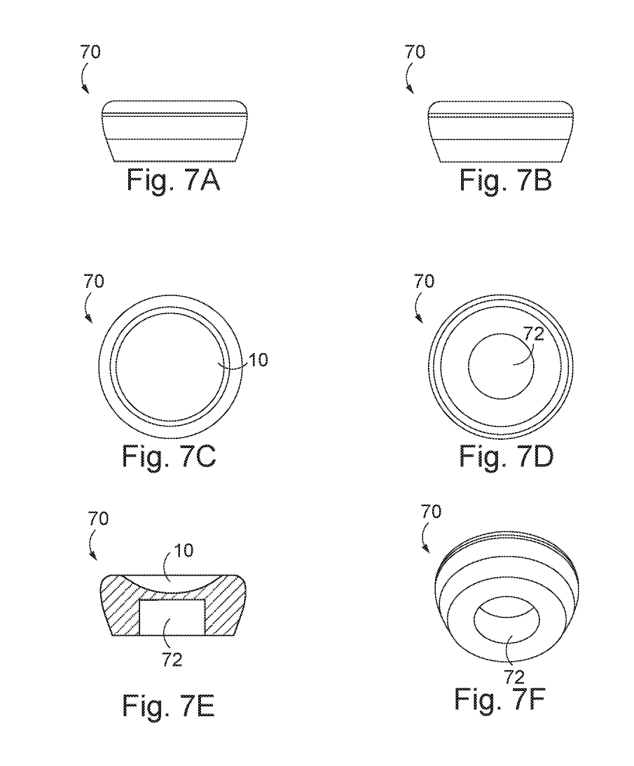

Referring also to the example embodiments of FIGS. 7A-7F, FIGS. 7A and 7B are a front and right views, respectively, illustrating a radial head component with cylindrical receptacle 70 in accordance with one exemplary embodiment of the radial head replacement prosthesis of the present disclosure. In some embodiments, the radial head component with cylindrical receptacle 70 may be one component of a radial head replacement prosthesis with a two-component design. The prosthesis and steps for implantation may generally be the same as that illustrated and described in FIGS. 1A to 1F with a few differences, which are described below. FIGS. 7C and 7D are top and bottom views, respectively, of radial head component with cylindrical receptacle 70. Illustrated are articular concavity 10, centered along the top surface, and cylindrical receptacle 72 centered along the bottom surface which may be highly polished and may provide a smooth cylindrical bearing surface for rotation. The cylindrical receptacle may have a rough surface. FIGS. 7E and 7F are a cross-sectional front view and bottom perspective view, respectively, further illustrating the radial head component with cylindrical receptacle 70 including articular concavity 10 and cylindrical receptacle 72. In some embodiments, the radial head component with cylindrical receptacle 70 may be made in multiple sizes to accommodate anatomical variations. Embodiments may include varying diameters and heights of the radial head component with cylindrical receptacle 70. The diameter and dimensions of the cylindrical receptacle may be consistent throughout all sizes in order to allow for modularity and compatibility.

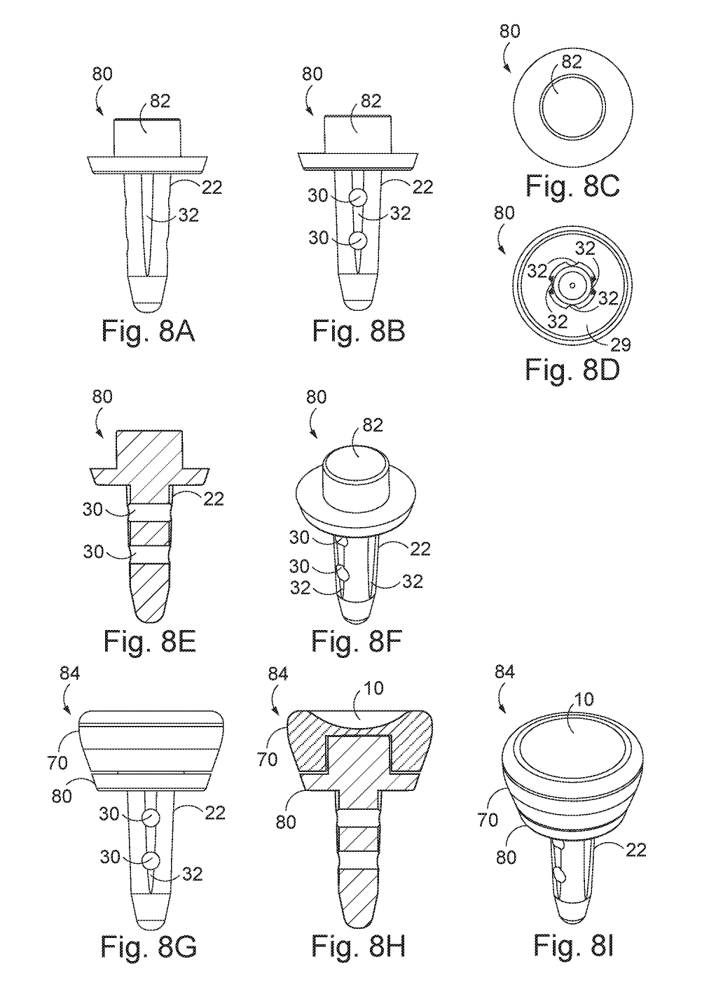

Referring also to the example embodiments of FIGS. 8A-8I, FIGS. 8A and 8B are a front and right views, respectively, illustrating a stem component with cylindrical extension 80 in accordance with one exemplary embodiment of the radial head replacement prosthesis of the present disclosure. In some embodiments, the stem component with cylindrical extension 80 may be one component of a radial head replacement prosthesis with a two-component design. The prosthesis and steps for implantation may generally be the same as that illustrated and described in FIGS. 2A to 2I with a few differences, which are described below. In one embodiment, the stem component with cylindrical extension 80 may include cylindrical extension 82. In some embodiments, cylindrical extension 82 may be highly polished and may provide for rotation when joined to the radial head component with cylindrical receptacle 70. FIGS. 8C and 8D are top and bottom views, respectively, further illustrating the stem component with cylindrical extension 80 including cylindrical extension 82, planar undersurface 29, and four of flute 32. FIGS. 8E and 8F are a cross-sectional front view and a perspective view, respectively, further illustrating the stem component including cylindrical extension 82, tapered stem 22, and interlocking screw hole 30 present in duplicate. FIGS. 8G and 8H are a right view and a cross-sectional front view, respectively, illustrating a radial head replacement prosthesis with cylindrical connection assembly 84 including radial head component with cylindrical receptacle 70 and stem component with cylindrical extension 80. In one embodiment, the radial head component with cylindrical receptacle 70 may be seated onto the stem component with cylindrical extension 80 to achieve a connection allowing rotation between the two components with cylindrical extension 80 inserting into the cylindrical receptacle 72. In some embodiments, Cylindrical extension 82 may be of slightly smaller diameter than cylindrical receptacle 72 to allow for rotation. FIG. 8I is a perspective view further illustrating the radial head replacement prosthesis with cylindrical connection assembly 84 after final seating of radial head component with cylindrical receptacle 70 onto stem component with cylindrical extension 80.

In some embodiments, the stem component with cylindrical extension 80 may be made in multiple sizes to accommodate anatomical variations. Embodiments include varying diameters and lengths of tapered stem 22. The diameter and dimensions of cylindrical extension 82 may be consistent throughout all sizes in order to allow for modularity and compatibility. The stem component with cylindrical extension 80 may therefore come in multiple sizing options to enable the surgeon to select appropriate size implants for each individual patient, as needed. Thus, patients with larger anatomy can receive a proportionally larger stem component with cylindrical extension.

The stem component with cylindrical extension may be manufactured to consist of metal or metal alloy including a high carbon cobalt chrome (CoCr) alloy. It may also be made of an alternate metal or metal alloy including stainless steel, titanium, or another suitable material including surgical-grade ceramics, plastics, rubber, or combination materials. It can be formed as cast, forged, milled or drilled through machining processes or a combination of methods. Standard machining techniques can be used during component manufacture including for creation of the cylindrical extension. The stem component with cylindrical extension may also be manufactured in constituent form and later joined or coupled through welding or other suitable techniques. Surfaces may be further smoothed to achieve a highly-polished finish during production through standard manufacturing techniques, as known in the art. The planar undersurface 29 and tapered stem 22, including each flute 32, may include a porous, textured, granular and/or beaded surface coating as known in the art. The cylindrical extension may have a rough surface.

The steps for implantation may generally be the same as illustrated and described in FIGS. 1A to 1F and FIGS. 2A to 2I, with a few differences described below. Following implantation of the stem component with cylindrical extension 80, an appropriately sized radial head component with cylindrical receptacle 70 may be selected based on the diameter and height of the excised native radial head. The radial head component with cylindrical receptacle 70 may be seated onto the stem component with cylindrical extension 80 achieving an inter-component connection which a allows smooth rotational movement. An advantage of the present embodiment may be that rotational movement between components is afforded which may be helpful to allow further rotation in the setting of pre-operative elbow stiffness.

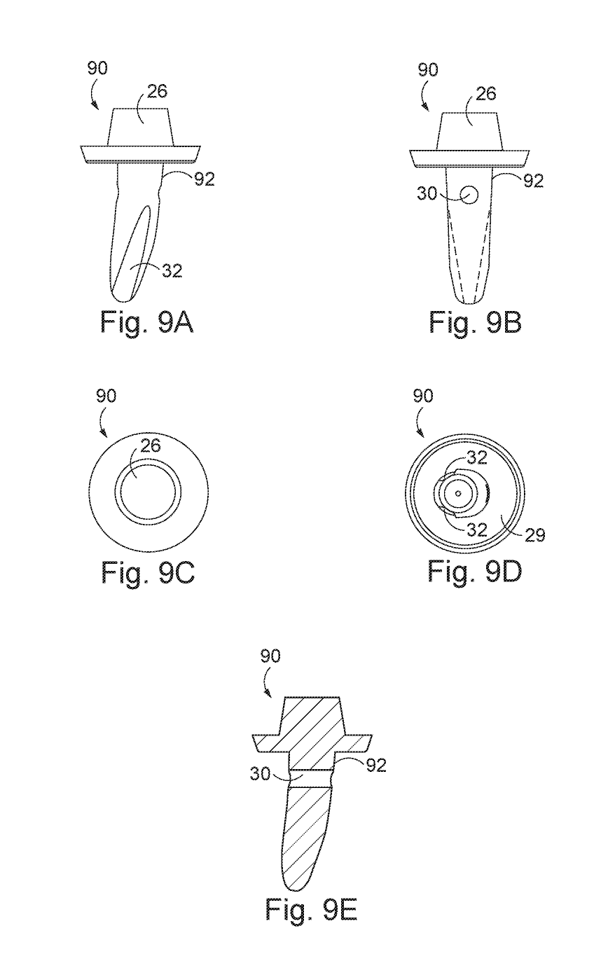

Referring also to the example embodiments of FIGS. 9A-9E, FIGS. 9A and 9B are a front and right views, respectively, illustrating a stem component with tapered extension and curved stem 90 in accordance with one exemplary embodiment of the radial head replacement prosthesis of the present disclosure. In some embodiments, the stem component with tapered extension and curved stem 90 may be one component of a radial head replacement prosthesis with a two-component design. The prosthesis and steps for implantation may generally be the same as that illustrated and described in FIGS. 2A to 2F with a few differences, which are described below. In some embodiments, the stem component with tapered extension and curved stem 90 may include tapered curved stem 92. The tapered curved stem may progressively narrow from its top diameter to its bottom diameter and may be curved in its front profile. This curved stem may be beneficial in the setting of increased curvature of the proximal radius and can accommodate bone in the proximal radius with and increased radius of curvature. The stem component with tapered extension and curved stem 90 may also include two of flute 32, and one interlocking screw hole 30. FIGS. 9C and 9D are top and bottom views, respectively further illustrating the stem component with Morse tapered extension and curved stem 90 including tapered extension 26, planar undersurface 29, and two of flute 32. FIG. 9E is a cross-sectional front view further illustrating the stem component with tapered extension and curved stem 90, including tapered curved stem 92 and one interlocking screw hole 30. The radial head component with tapered receptacle 9 of FIGS. 1A to 1F may be seated onto the stem component with tapered extension and curved stem 90 during the operative procedure.

The steps for implantation may generally be the same as illustrated and described in FIGS. 1A to 1F and FIGS. 2A to 2I, with a few differences described below. Following surgical preparation including removal of the radial head, surgical drills or broach/es may be used to prepare the medullary canal for implantation of the stem component with tapered extension and curved stem 90 to create a medullary channel to accommodate curved tapered stem 92 with the broaches guided along a curved path creating the curved medullary channel. The stem component with tapered extension and curved stem 90 may then be inserted and seated into the medullary canal with the planar undersurface 29 abutting the osteotomized bone of the radial cortex. In some embodiments, at least one interlocking screw hole is drilled to align with one or more interlocking screw holes 30, with the aid of intraoperative fluoroscopic guidance. In a preferred embodiment, a single interlocking screw hole is drilled to align with interlocking screw hole 30 for implant fixation. A short screw may then be inserted from the outer cortex entering and engaging the interlocking screw hole and may extend beyond the interlocking screw hole but not beyond the far cortex. Intraoperative fluoroscopic image guidance can aid localization and drilling of the cortical hole to accommodate the screw to engage the interlocking screw hole within the tapered curved stem. Following implantation of the stem component with tapered extension and curved stem 90, an appropriately sized radial head component with tapered receptacle 9 may be selected based on the diameter and height of the excised native radial head. The selected radial head component with tapered receptacle may be firmly seated onto the stem component with tapered extension and curved stem 90 to achieve an interference fit with tapered extension 26 inserting into the tapered receptacle 12. A surgical tamp may be gently tapped along the top periphery of the radial component to further aid final seating of the radial head component implant. An advantage of the present embodiment is that tapered curved stem 92 may be beneficial in for implantation in patients with increased curvature of the proximal radius to accommodate native bone curvature.

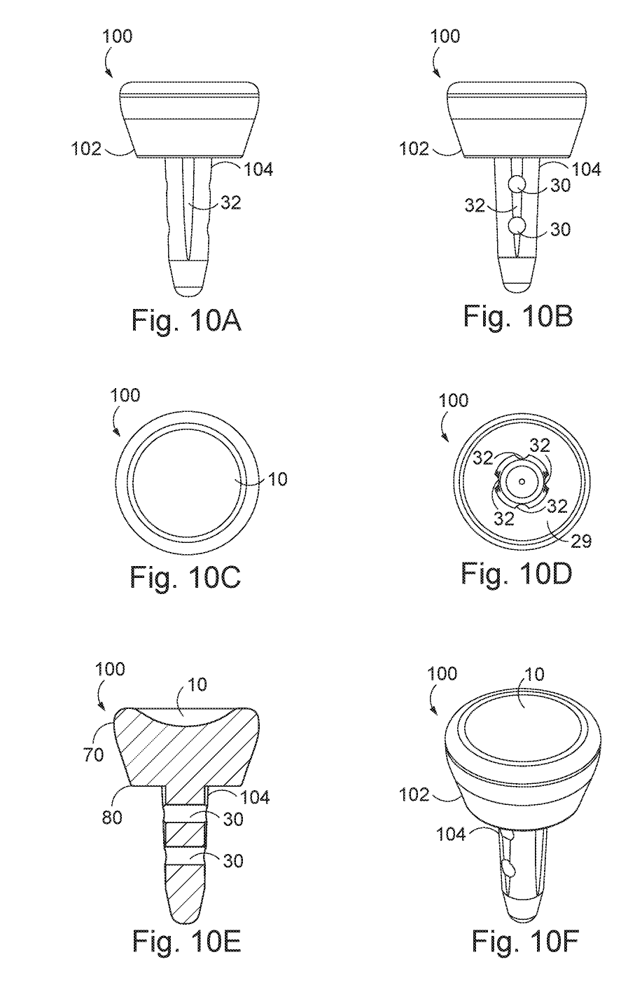

Referring also to the example embodiments of FIGS. 10A-10F, FIGS. 10A and 10B are front and right views, respectively, illustrating one-piece radial head replacement prosthesis 100 in accordance with one exemplary embodiment of the radial head replacement prosthesis of the present disclosure. The prosthesis and steps for implantation may generally be the same as that illustrated and described in FIGS. 1A to 1F and FIGS. 2A to 2I with a few differences, which are described below. The one-piece radial head replacement prosthesis 100 may include upper aspect 102, lower aspect with tapered stem 104, four of flute 32, and two of interlocking screw hole 30. The radial head and stem aspects of the prosthesis of the present embodiment may be coupled together into a single component during manufacture, as known in the art. Standard machining techniques can be used during manufacture for creation of the one-piece radial head replacement prosthesis. The upper aspect 102 and lower aspect with tapered stem 104 may be manufactured with forging or casting techniques or manufactured separately and joined secondarily through welding or other suitable techniques as known in the art including interference fit, a threaded extension, etc. FIGS. 10C and 10D are top and bottom views, respectively, further illustrating the one-piece radial head replacement prosthesis including articular concavity 10, articular planar undersurface 29 and four of flute 32. FIGS. 10E and 10F are a cross-sectional front view and a perspective view, respectively, further illustrating the one-piece radial head replacement prosthesis 100 including articular concavity 10 along the top surface.

The steps for implantation may generally be the same as illustrated and described in FIGS. 1A to 1F and FIGS. 2A to 2I, with a few differences described below. Following surgical preparation including removal of the radial head and preparation of the medullary canal, a one-piece radial head replacement prosthesis 100 may be selected based on the diameter and height of the native radial head that has been removed with the stem length matching the prepared medullary canal length. The one-piece radial head replacement prosthesis 100 may be inserted into the medullary canal. Next one or more interlocking screw holes may be drilled to align with each of the interlocking screw holes 30, with the aid of intraoperative fluoroscopic guidance. In a preferred embodiment, two interlocking screw holes may be drilled to align with each interlocking screw hole 30, with the aid of intraoperative fluoroscopic guidance. In this example, two short screws may then be inserted from the outer cortex entering and engaging the interlocking screw holes and may extend beyond the interlocking screw holes but not beyond the far cortex. Intraoperative fluoroscopic image guidance can aid localization and drilling of the cortical holes to accommodate the screws to engage the interlocking screw holes. An advantage of the present embodiment is that the one-piece radial head replacement prosthesis may reduce initial manufacturing time depending and mode of manufacture, as the radial head replacement prosthesis consists of a single component instead of two constituent components.

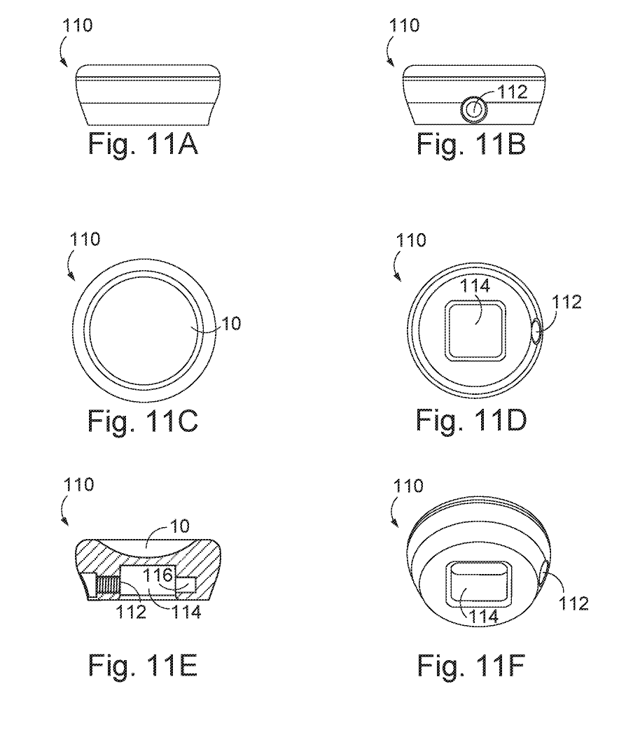

Referring also to the example embodiments of FIGS. 11A-11F, FIGS. 11A and 11B are a front and right views, respectively, illustrating a radial head component with inter-component threaded screw hole 110 in accordance with one exemplary embodiment of the radial head replacement prosthesis of the present disclosure. In some embodiments, the radial head component with inter-component threaded screw hole 110 may be one component of a radial head replacement prosthesis with a two-component design. The prosthesis and steps for implantation may generally be the same as that illustrated and described in FIGS. 1A to 1F with a few differences, which are described below. Along the right side of the radial head component with inter-component threaded screw hole 110 may be recessed inter-component threaded screw hole 112. The recessed inter-component threaded screw hole can accept a screw which can be tightened and seated and can also allow the screw head to not extend beyond the recess. FIGS. 11C and 11D are top and bottom views, respectively, illustrating the radial head component with inter-component threaded screw hole 110 including articular concavity 10, which may be centered along the top surface. Along the bottom of the radial head component with inter-component threaded screw hole 110 is cube shaped receptacle 114 which may allow for a shape fit and resistance to rotational motion between components when mated to a similar cube shaped extension of a matching prosthetic stem components, as will be described. The cube shaped receptacle 114 may have a rough surface. FIGS. 11E and 11F are a cross-sectional front view and bottom perspective view, respectively, further illustrating the radial head component with inter-component threaded screw hole 110 including articular concavity 10, recessed inter-component threaded screw hole 112, and screw hole extension 116. In some embodiments, screw hole extension 116 is unthreaded and may allow an inserted screw to extend beyond recessed inter-component threaded screw hole 112, past the cube shaped receptacle 114 and into the screw hole extension. The radial head component with inter-component threaded screw hole 110 may be made in multiple sizes to accommodate anatomical variations. Embodiments include varying diameters and heights of the radial head component with inter-component threaded screw hole 110. The diameter and dimensions of the cube shaped receptacle and recessed inter-component threaded screw hole and screw hole extension may be consistent throughout all sizes in order to allow for modularity and compatibility.

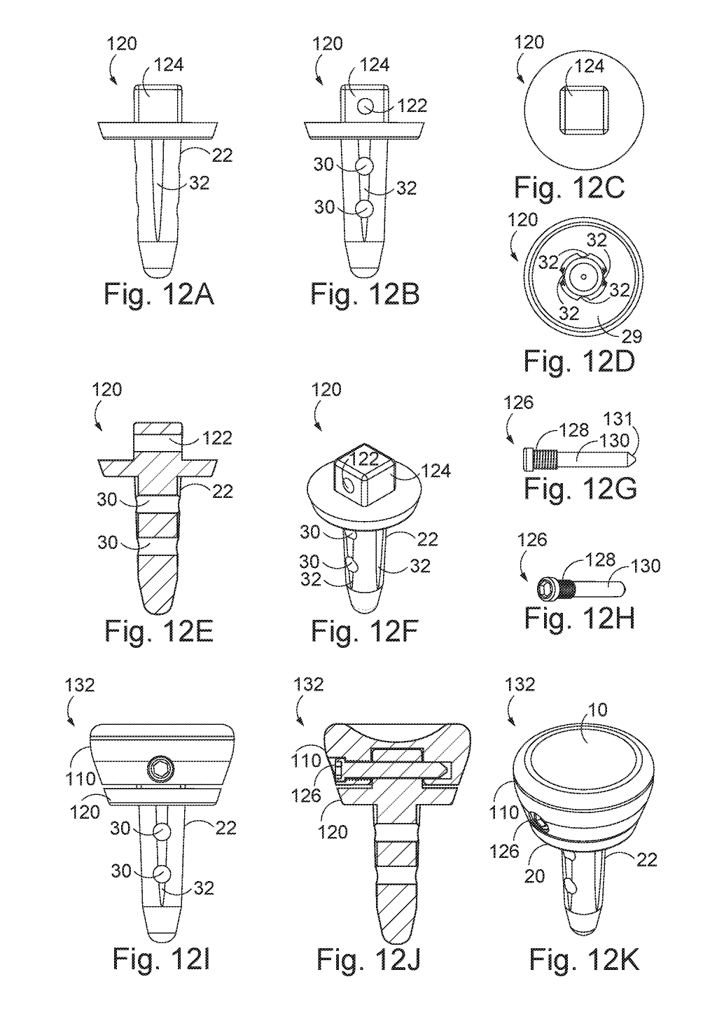

Referring also to the example embodiments of FIGS. 12A-12K, FIGS. 12A and 12B are a front and right views, respectively, illustrating a stem component with inter-component screw hole 120 in accordance with one exemplary embodiment of the radial head replacement prosthesis of the present disclosure. The stem component with inter-component screw hole 120 may be one component of a radial head replacement prosthesis with a two-component design. The prosthesis and steps for implantation may generally be the same as that illustrated and described in FIGS. 2A to 2I with a few differences, which are described below. In some embodiments, the stem component with inter-component screw hole 120 includes cube shaped extension 124. In some embodiments, cube shaped extension 124 may provide a connection point for the radial head component with inter-component screw hole 110. The cube shaped extension 124 of stem component with inter-component screw hole 120 may insert into the cube shaped receptacle 114 of radial head component with inter-component screw hole 110 to achieve a shape fit. The cube shaped extension 124 may have a rough surface. Stem inter-component non-threaded screw hole 122 may be included along cube shaped extension 124. Also included in stem component with inter-component screw hole 120 are tapered stem 22, four of flute 32, and interlocking screw hole 30 in duplicate. FIGS. 12C and 12D are top and bottom views, respectively, further illustrating the stem component with inter-component screw hole 120 including cube shaped extension 124, four of flute 32 and planar undersurface 29. FIGS. 12E and 12F are a cross-sectional front view and a perspective view, respectively, further illustrating the stem component with inter-component screw hole 120, including stem inter-component non-threaded screw hole 122, cube shaped extension 124, tapered stem 22, and interlocking screw hole 30 present in duplicate. FIGS. 12G and 12H are front and perspective views, respectively, illustrating an inter-component partially threaded screw 126 which includes threaded aspect 128, non-threaded aspect 130 and tapered aspect 131. Inter-component partially threaded screw 126 may help to join and retain radial head component with inter-component screw hole 110 on stem component with inter-component screw hole 120 following completed placement. FIGS. 121 and 12J are a right view and a cross-sectional front view, respectively, illustrating a radial head replacement prosthesis with inter-component screw connection assembly 132 including radial head component with inter-component screw hole 110, stem component with inter-component screw hole 120 and inter-component partially threaded screw 126. The radial head component with inter-component screw hole 110 may be seated onto the stem component with inter-component screw hole 120 to achieve a shape fit with the cube shaped extension inserting into the cube shaped receptacle. Following seating, inter-component partially threaded screw 126 may be inserted into recessed inter-component threaded screw hole 112, then extending through stem inter-component non-threaded screw hole 122 and into screw hole extension 116, to further retain component seating. FIG. 12K is a perspective view further illustrating final seating of radial head component with inter-component screw hole 110 onto stem component with inter-component screw hole 120 and inter-component partially threaded screw 126.

The inter-component partially threaded screw may be manufactured to consist of metal or metal alloy including a high carbon cobalt chrome (CoCr) alloy, as known in the art. It may also be made of an alternate metal or metal alloy including stainless steel, titanium, or another suitable material including surgical-grade ceramics, plastics, rubber, or combination materials. It can be formed as cast, forged, milled or drilled through machining processes or a combination of methods. Standard machining techniques can be used during component manufacture.

The steps for implantation may generally be the same as illustrated and described in FIG. 1A to 1F and FIGS. 2A to 2I, with a few differences described below. Following implantation of the stem component with inter-component screw hole 120, an appropriately sized radial head component with inter-component screw hole 110 may be selected based on the diameter and height of the excised native radial head. The radial head component with inter-component screw hole 110 may be oriented to line up with and seat onto the stem component with Morse tapered extension and anti-rotation pegs 60 with cube shaped extension 124 inserting and seating into the cube shaped receptacle 114 forming a shape fit and with recessed inter-component threaded screw hole 112 aligning with stem inter-component non-threaded screw hole 122 to allow for inter-component partially threaded screw 126 to be fully inserted and secured. Next, an inter-component partially threaded screw 126 may be inserted into recessed inter-component threaded screw hole 112, extending through stem inter-component non-threaded screw hole 122 and into screw hole extension 116, to further retain component seating. An advantage of the present embodiment is that a capability for placement of an inter-component screw may be beneficial in the setting of elbow joint laxity or ligamentous compromise to help further maintain component seating.

While the above description contains much specificity, this should not be construed as limitations on the scope, but rather an exemplification of one or more exemplary embodiments as detailed. Multiple additional configurations are possible of the radial head replacement prosthesis. The porous coating along the tapered stem and tapered curved stem may be omitted from any embodiment but is currently preferred to allow for osseous integration along the implant. The porous coating may also be omitted from the flutes, but it is presently preferred to retain the porous coating along the flutes. Inclusion of a different number of interlocking screw holes including one, two, three or more is possible in all embodiments, as well as alteration of the stem taper angle, and tapered stem length and curved stem length in the described embodiments. Accordingly, it will be appreciated that any number of interlocking screw holes is possible within the scope of the present disclosure. Inclusion of a tapered curved stem with an increased or decreased degree of taper from its upper aspect to its lower aspect, or a stem with no taper at all, is possible for all embodiments. Inclusion of a longer, or shorter, tapered stem or tapered curved stem, is possible for all embodiments. The tapered or tapered curved stem may be shortened or lengthened. The stem may be non-tapered in any embodiment. One or both anti-rotation pegs may omitted or additional anti-rotation pegs may be added or anti-rotation pegs of a different shape may be included, in each case with matching modifications to the associated radial head component anti-rotation peg receptacle/s. Accordingly, it will be appreciated that any number of anti-rotation pegs is possible within the scope of the present disclosure. Flutes may be present in any embodiment in a different configuration including more or less flutes, or flutes with a different contour or geometry or the flutes may be omitted. For example, zero, one, two or three flutes or more along any stem may be present in any embodiment. Accordingly, it will be appreciated that any number of flutes is possible within the scope of the present disclosure. Inclusion of flutes may be preferred to further aid implantation and provide for additional rotational stability. The interlocking screw holes may include threads in any embodiment to allow screws to engage the threaded interlocking screw holes for added fixation capability. The interlocking screw holes may be included or omitted from any embodiment and additional interlocking screw holes may be included in any embodiment including a combination of interlocking screw holes and interlocking screw holes which are threaded. The interlocking screw holes may be in differing trajectories from each other, including in perpendicular orientations. The interlocking screw holes along the tapered stem may be located higher or lower along the stem and may be in any trajectory including an oblique orientation in any embodiment. The interlocking screw holes may also include a tapered or filleted edge along their outer edge to further aid screw insertion. All embodiments may include or exclude an alignment indentation or have the alignment indentation present along an alternate surface including along any surface along any radial head replacement embodiment. The alignment indentation may also be shortened or enlarged in any dimension. The tapered receptacle of radial head component with tapered receptacle 9 and tapered extension of stem component with tapered extension 20 may be modified to accommodate an alternate geometric shape or connection type. The stem inter-component non-threaded screw hole and/or screw hole extension can be threaded to allow further threaded component engagement. All embodiments can be modified to allow for inter-component screw connection including alternate types of screws. For example, the radial head replacement prosthesis with taper assembly may also have capability for placement of an inter-component screw. These alternate connections may also be utilized all embodiments of the radial head replacement prosthesis of the present disclosure. The tapered connection of the radial head replacement prosthesis with taper assembly may be reoriented so that the tapered extension is included along the radial head component and the tapered receptacle is included along the stem component. The cylindrical connection of the radial head replacement prosthesis with cylindrical connection assembly may be reoriented so that the cylindrical extension is included along the radial head component and the cylindrical receptacle is included along the stem component.

The radial head replacement prosthesis of the present disclosure may each be made of metal or metal alloy including high carbon cobalt chrome (CoCr) alloy, stainless steel, titanium or aluminum, surgical-grade ceramics, ceramic-coated metal, oxidized metals, polyethylene, rubber, or another suitable material, or combination of suitable materials. They may be manufactured as cast or using additional standard techniques including injection molding, forging, bending during the machining process, and can be manufactured in segments and welded, plastic welded or otherwise joined, e.g., with an inference fit.

Although the present disclosure has been described with reference to particular examples and exemplary embodiments, it should be understood that the foregoing description is in no manner limiting. For example, although the present disclosure has been described with reference to particular materials, manufacturing methods and joining methods, it should be understood that other suitable materials such as for example plastics, rubber or ceramics, other manufacturing methods such as for example injection molding, laser cutting or alternate machining methods, and other joining methods such as for example friction fitting may be encompassed by the present disclosure. Moreover, the features described herein may be used in any combination.

While one or more example implementations have been described using a radial head replacement prosthesis for a human, it will be appreciated that the present disclosure may be adapted for use for animals as well. As such, the description of a radial head replacement prosthesis for a human should be used as example only and not to otherwise limit the scope of the disclosure.

What is claimed is:

1. A radial head replacement prosthesis comprising:
a radial head component with a concave bearing surface configured to articulate with a capitelum of a distal humerus; and
a stem component with at least one interlocking screw hole configured to accept a screw.

2. The radial head replacement prosthesis of claim 1, further comprising:
a tapered connection configured to couple said radial head component and said stem component.

3. The radial head replacement prosthesis of claim 1, wherein said stem component includes an alignment indentation.

4. The radial head replacement prosthesis of claim 1, wherein said radial head component includes one or more anti-rotation holes.

5. The radial head replacement prosthesis of claim 1, wherein said stem component includes one or more anti-rotation pegs.

6. The radial head replacement prosthesis of claim 1, further comprising:
a cylindrical connection configured to couple said radial head component and said stem component.

7. The radial head replacement prosthesis of claim 1, wherein said stem component is a tapered curved stem.

8. The radial head replacement prosthesis of claim 1, wherein said radial head component and said stem component are a single component.

9. The radial head replacement prosthesis of claim 1, wherein said radial head component and said stem component are each configured to accommodate an inter-component partially threaded screw configured to couple said radial head component and said stem component.

10. The radial head replacement prosthesis of claim 1, wherein said stem component includes at least one surface with a porous coating.

11. A radial head replacement prosthesis comprising:
a radial head component comprising:
a concave bearing surface configured to articulate with a capitelum of a distal humerus, and
a tapered receptacle; and
a stem component comprising:
at least one interlocking screw hole configured to accept a screw, and
a tapered extension configured to couple to said tapered receptacle of said radial head component.

12. The radial head replacement prosthesis of claim 11, wherein said stem component includes an alignment indentation.

13. The radial head replacement prosthesis of claim 11, wherein said radial head component includes one or more anti-rotation holes.

14. The radial head replacement prosthesis of claim 11, wherein said stem component includes one or more anti-rotation pegs.

15. The radial head replacement prosthesis of claim 11, further comprising:
a cylindrical connection configured to couple said radial head component and said stem component.

16. The radial head replacement prosthesis of claim 11, wherein said stem component is a tapered curved stem.

17. The radial head replacement prosthesis of claim 11, wherein said radial head component and said stem component are a single component.

18. The radial head replacement prosthesis of claim 11, wherein said radial head component and said stem component are each configured to accommodate an inter-component partially threaded screw configured to couple said radial head component and said stem component.

19. The radial head replacement prosthesis of claim 11, wherein said stem component includes at least one surface with a porous coating.

20. A radial head replacement prosthesis comprising:
a radial head component comprising:
a concave bearing surface configured to articulate with a capitulum of a distal humerus, and
a cylindrical receptacle; and
a stem component comprising:
at least one interlocking screw hole configured to accept a screw, and
a cylindrical extension configured to couple to said cylindrical receptacle of said radial head component.

21. The radial head replacement prosthesis of claim 20, wherein said radial head component includes one or more anti-rotation holes.

22. The radial head replacement prosthesis of claim 20, wherein said stem component includes one or more anti-rotation pegs.

23. The radial head replacement prosthesis of claim 20, wherein said stem component is a tapered curved stem.

24. The radial head replacement prosthesis of claim 20, wherein the stem component includes at least one surface with a porous coating.
